(19) United States
(12) Patent Application Publication
(10) Pub. No.: US 2024/0145056 A1
(43) Pub. Date: May 2, 2024

(54) SYSTEM AND PROCESS FOR FEATURE EXTRACTION FROM THERAPY NOTES

(71) Applicant: Monterad/b/a Forta, San Francisco, CA (US)

(72) Inventors: Jenish MAHARJAN, San Francisco, CA (US); Anurag GARIKIPATI, San Francisco, CA (US); Navan Preet SINGH, San Francisco, CA (US); Madalina CIOBANU, San Francisco, CA (US); Qingqing MAO, San Francisco, CA (US)

(21) Appl. No.: 18/496,525

(22) Filed: Oct. 27, 2023

Related U.S. Application Data
(60) Provisional application No. 63/419,756, filed on Oct. 27, 2022.

(51) Int. Cl. G16H 15/00; G16H 10/60; G16H 20/30; G16H 20/70; G16H 80/00

(52) U.S. Cl. CPC G16H 15/00; G16H 10/60; G16H 20/30; G16H 20/70; G16H 80/00

(57) ABSTRACT

A method implemented via a computing device. The method may include receiving, by the computing device, treatment data associated with a subject. The treatment data associated with the subject may include behavior data, mood data, emotions data, goals data, instruction data, or combinations thereof. The method may also include evaluating, by the computing device, the treatment data associated with the subject via a therapy notes (TN) model. The TN model may evaluate the treatment data associated with the subject to generate one or more therapy note features.

### CROSS-REFERENCE TO RELATED APPLICATIONS

This application claims priority to U.S. Application Ser. No. 63/419,756 filed Oct. 27, 2022, entitled System and Process for Feature Extraction From Session Notes for Qualitative Analysis, which is incorporated herein by reference in its entirety.

### TECHNICAL FIELD

The present disclosure relates to methods and systems pertaining to the use of artificial intelligence for evaluating therapy data associated with an individual with a neurodevelopmental disorder (NDD) such as autism spectrum disorder (ASD).

### BACKGROUND

NDDs are generally associated with impaired neurological development, often leading to abnormal brain function, and exhibited as emotional, learning, behavioral, and/or cognitive aberrances that can affect sensory systems, motor systems, speech, and language. An example of an NDD includes ASD. Other examples of an NDD include attention deficit hyperactivity disorder (ADHD), other specified ADHD, unspecified ADHD; motor disorders, developmental coordination disorder, stereotypic movement disorder, tic disorders, Tourette's disorder or syndrome, persistent (chronic) motor or vocal tic disorder, provisional tic disorder, other specified tic disorder, unspecified tic disorder; cerebral palsy; Rett syndrome; intellectual disabilities, intellectual developmental disorder, global developmental delay, unspecified intellectual disability, unspecified intellectual developmental disorder; communication disorders, language disorder, speech sound disorder or phonological disorder, childhood-onset fluency disorder or stuttering; social or pragmatic communication disorder, unspecified communication disorder; specific learning disorder; other NDDs, other specified NDD, unspecified NDD; or combinations thereof

Supervisory therapists and therapists use therapy notes (for example, notes created during, in the course of, or associated with one or more therapy sessions) as a tool to document and evaluate the progress of a patient, for example, that relate to the goals and objectives outlined in their treatment plans over the course of treatment, such as applied behavior analysis (ABA) treatment or therapy. For example, the patient may be characterized as having an NDD such as ASD, ADHD, other specified ADHD, unspecified ADHD; motor disorders, developmental coordination disorder, stereotypic movement disorder, tic disorders, Tourette's disorder or syndrome, persistent (chronic) motor or vocal tic disorder, provisional tic disorder, other specified tic disorder, unspecified tic disorder; cerebral palsy; Rett syndrome; intellectual disabilities, intellectual developmental disorder, global developmental delay, unspecified intellectual disability, unspecified intellectual developmental disorder; communication disorders, language disorder, speech sound disorder or phonological disorder, childhood-onset fluency disorder or stuttering; social or pragmatic communication disorder, unspecified communication disorder; specific learning disorder; other NDDs, other specified NDD, unspecified NDD; or combinations thereof. The terms “treatment” and “therapy” may be utilized interchangeably in the context of this disclosure. Therapy notes can also be required for some payers, for example, to obtain payment or reimbursement by a health care insurance company (e.g., health insurer). For instance, therapy notes may not be required to submit a health insurance claim, but may be requested by a payer (e.g., a health insurer) during an audit. In most cases, the provider who renders a session (e.g., therapy session, treatment session), a therapist (such as a certified therapist) in most cases, completes a therapy note form, including all required information. The authorized ABA supervisor, a supervisory therapist for instance (such as a certified supervisory therapist), then reviews the therapy note to ensure that all the required elements are present and proper. Review and evaluation of the therapy note by the authorized ABA supervisor is a demanding task, especially considering the lack of supervisory therapists and therapists in general, especially in light of a high demand for ABA therapy services and low availability of ABA therapy providers. The system disclosed herein is a tool that aims at streamlining this process of reviewing and evaluating therapy notes.

Given the complex and challenging nature of evaluating therapy notes, there is an ongoing need to develop and provide systems and methods for the evaluation of therapy notes.

### BRIEF DESCRIPTION

In some embodiments disclosed herein is a method implemented via a computing device. The method may comprise receiving, by the computing device, treatment data associated with a subject. The treatment data associated with the subject may comprise behavior data, mood data, emotions data, goals data, instruction data, or combinations thereof. The method may also comprise evaluating, by the computing device, the treatment data associated with the subject via a therapy notes (TN) model. The TN model may be configured to evaluate the treatment data associated with the subject to generate one or more therapy note features.

Also, in some embodiments disclosed herein is a system that may comprise a computing device. The computing device may comprise a processor and a non-transitory computer-readable medium. The non-transitory computer-readable medium may include instructions configured to cause the processor to implement a TN model. The TN model, when implemented via the processor, may cause the computing device to receive treatment data associated with a subject having a neurodevelopmental disorder (NDD). The treatment data associated with the subject may comprise behavior data, mood data, emotions data, goals data, instruction data, or combinations thereof. The TN model, when implemented via the processor, may also cause the computing device to evaluate the data associated with the subject via the TN model, wherein the TN model is configured to evaluate the treatment data associated with the subject to generate one or more therapy note features. The system may also comprise an application in communication with the computing device. The application may be configured to receive the one or more therapy note features from the computing device.

Also, in some embodiments disclosed herein is a method implemented via a computing device. The method may comprise receiving, by the computing device, training data associated with a plurality of subjects. At least a portion of the subjects may be individuals characterized as having an NDD. The training data associated with each of the plurality of subjects may comprise behavior data, mood data, emotions data, goals data, instruction data, or combinations thereof. The method may also comprise processing the training data associated with the plurality of subjects to yield a TN model. The TN model may be configured to evaluate treatment data associated with a subject to generate one or more therapy note features.

Also, in some embodiments disclosed herein is a method implemented via a computing device. The method may comprise generating, by the computing device, one or more therapy note features based upon treatment data associated with subject via a TN model. The treatment data associated with the subject may comprise behavior data, mood data, emotions data, goals data, instruction data, or combinations thereof. The method may also comprise inputting the therapy note features, by the computing device, into a therapy note.

Also, in some embodiments disclosed herein is a system comprising a computing device. The computing device may comprise a processor and a non-transitory computer-readable medium. The non-transitory computer-readable medium may include instructions configured to cause the processor to implement a TN model. The TN model, when implemented via the processor, may cause the computing device to generate, by the computing device, one or more therapy note features based upon treatment data associated with subject via the TN model. The treatment data associated with the subject may comprise behavior data, mood data, emotions data, goals data, instruction data, or combinations thereof. The TN model, when implemented via the processor, may also cause the computing device to input the therapy note features, by the computing device, into a therapy note. The system may also comprise an application in communication with the computing device. The application may be configured to receive the one or more therapy note features from the computing device.

Also, in some embodiments disclosed herein is a method implemented via a computing device. The method may comprise receiving, by the computing device, training data associated with a plurality of subjects. At least a portion of the subjects may be individuals characterized as having an NDD. The training data associated with each of the plurality of subjects may comprise behavior data, mood data, emotions data, goals data, instruction data, or combinations thereof. The method may also comprise processing the training data associated with the plurality of subjects to yield a TN model. The TN model may be configured to generate, by the computing device, one or more therapy note features based upon treatment data associated with subject via the TN model. The treatment data associated with the subject may comprise behavior data, mood data, emotions data, goals data, instruction data, or combinations thereof.

### BRIEF DESCRIPTION OF DRAWINGS

FIG. 1 is a schematic of a system and process according to one or more embodiments disclosed herein.

FIG. 2 is a schematic of a rule-based evaluation system.

FIG. 3 is a schematic of a natural language processing/machine learning evaluation system.

FIG. 4 is an example of an interface which may be used in accordance with one or more embodiments disclosed herein.

FIG. 5 is an example of natural language processing/machine learning evaluation system development.

FIG. 6 is a flow-chart of an example of a process of therapy note evaluation according to one or more embodiments disclosed herein.

FIG. 7 is a flow-chart of an example of a process for speech to text input evaluation according to one or more embodiments disclosed herein.

FIG. 8 is a flow-chart of an example of a process for audio sensing input evaluation according to one or more embodiments disclosed herein.

FIG. 9 is a flow-chart of an example of a video sensing input evaluation according to one or more embodiments disclosed herein.

FIG. 10 is a schematic representation of a computing system by way of which a machine-learning model may be employed.

FIG. 11 is a schematic representation of a machine-learning model.

### DETAILED DESCRIPTION

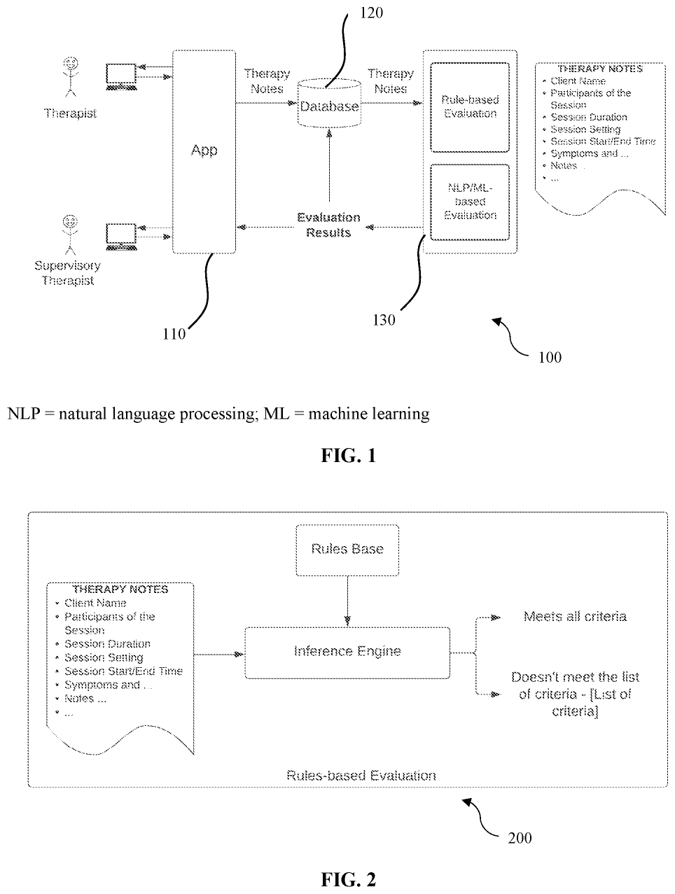

FIG. 1 illustrates a system and process associated with a therapy note. Generally, as used herein, the term “therapy note” is intended to refer, in whole or in part, to a collection of data that is obtained by a therapy provider in the course of one or more therapy sessions, regardless of the particular type of data or the form or format in which the data are recorded. Thus, reference to a therapy note, or therapy notes, should be understood as pertaining to the collection of data, regardless of the particular data or form or format thereof. In FIG. 1, a therapist documents the information onto a therapy note. A therapist can be any suitable individual that has undergone required training to deliver treatment or therapy to a patient/subject with a neurodevelopmental disorder (NDD) and passed a competency exam to demonstrate the required knowledge. Nonlimiting examples of NDDs include disorders on the autism spectrum or autism spectrum disorder (ASD), attention deficit hyperactivity disorder (ADHD), other specified ADHD, unspecified ADHD; motor disorders, developmental coordination disorder, stereotypic movement disorder, tic disorders, Tourette's disorder or syndrome, persistent (chronic) motor or vocal tic disorder, provisional tic disorder, other specified tic disorder, unspecified tic disorder; cerebral palsy; Rett syndrome; intellectual disabilities, intellectual developmental disorder, global developmental delay, unspecified intellectual disability, unspecified intellectual developmental disorder; communication disorders, language disorder, speech sound disorder or phonological disorder, childhood-onset fluency disorder or stuttering; social or pragmatic communication disorder, unspecified communication disorder; specific learning disorder; other NDDs, other specified NDD, unspecified NDD; or combinations thereof. The therapist may or may not be certified, such as board certified. For example, some governing bodies (e.g., boards) may require the therapists to be certified, while other governing bodies may allow therapists to practice after completing training and passing a competency exam to become therapists, and further certification (such as board certification) may not be required). Nonlimiting examples of therapists as may provide therapy according to this disclosure include any suitable therapists, registered therapists, behavior therapists, registered behavior therapists, international behavior therapists, registered international behavior therapists, behavior technicians, registered behavior technicians, applied behavior analysis technicians, registered applied behavior analysis technicians, autism therapists, registered autism therapists, autism technicians, registered autism technicians, autism practitioners, registered autism practitioners, occupational therapists, registered occupational therapists, physical therapists, registered physical therapists, speech therapists, registered speech therapists, speech pathologists, registered speech pathologists, and the like, or combinations thereof, as well as assistants to there. The therapist may be a parent of the patient, a relative of the patient, or any suitable caregiver for the patient. When the information has been input, the therapy note may be then passed on to a supervisory therapist who reviews and evaluates the contents of the therapy note. Nonlimiting examples of supervisory therapists include any suitable supervisory therapists, certified supervisory therapists, board certified supervisory therapists, psychologists, certified psychologists, board certified psychologists, supervisory autism practitioners, certified supervisory autism practitioners, board certified supervisory autism practitioners, supervisory autism service practitioners, certified supervisory autism service practitioners, board certified supervisory autism service practitioners, qualified autism services practitioner-supervisor, certified qualified autism services practitioner-supervisors, board certified qualified autism services practitioner-supervisors, qualified behavior analysts, certified qualified behavior analysts board certified qualified behavior analysts, professionals, certified behavior professionals, board certified behavior professionals, behavior analysts, certified behavior analysts, board certified behavior analysts, international behavior analyst, certified international behavior analyst, board certified international behavior analyst, autism professionals, certified autism professionals, board certified autism professionals, autism specialists, certified autism specialists, board certified autism specialists, and the like, or combinations thereof. The supervisory therapist may or may not be licensed. A supervisory therapist checks and verifies the content of the therapy note. In addition to just checking the validity or completeness of all the elements of the therapy note, a supervisory therapist performs a qualitative review of the narrative summary of the session. While these checks are most often objective, the time taken to evaluate such therapy notes might amount to multiple hours every week for a supervisory therapist depending on the number of individuals they supervise. The proposed system provides for the automated evaluation of therapy notes in the process in order to cut down the time and effort that a supervisory therapist spends evaluating the therapy notes. The proposed system may also be effective to reduce the chances of human error. Some of the concerns that the system may be effective to address include, but are not limited to:
Minimizing the workload for supervisory therapists by automating the therapy note evaluation process.
Minimizing the chances of possible human errors.
Providing a more objective method of therapy note evaluation.

In the embodiment of FIG. 1, the system 100 generally comprises a therapy note application 110 (e.g., the “App”). Generally, the therapy note application 110 may be the primary interface through which the users, for example, a therapist or supervisory therapist, may be able to record, review, update, evaluate and approve therapy notes. For example, the therapy notes application 110 may provide an interface where the user may be able to note the information for the sessions conducted for a client. Therapists may be able to complete therapy notes after every session and update the therapy notes if requested for update. In various embodiments, the therapy notes may be derived from or associated with applied behavior analysis (ABA) therapy, speech therapy, language therapy, physical therapy, occupational therapy, or combinations thereof. The supervisory therapist may be able to view completed therapy notes and/or a summary of the system's evaluation of the therapy note, and to request updates from the therapist, if necessary. The therapy notes application 110 may include (or be in communication with) a data store where all the completed therapy notes along with the updates are stored.

Additionally, in some embodiments, the therapy note application 110 may communicate with a database 120 utilized to store data once collected during a session. The data collected from the session may be processed and stored in the database 120 prior to formatting, processing, and being fed into a therapy note evaluation engine 130, as will be disclosed herein. Data collected from a therapy session may be broken down into sub-components based on the field within the collected note, as illustrated in FIG. 1. The data may be converted to specific formats based on the data type of the specific field. As an example, the Client Name may be stored as a set of characters (for example, a string type variable). As another example, the Session Date can be stored as a date (for example, a date-time variable). This mapping of session note information to database inputs may exactly match the fields from the therapy note data that is collected, for example, during a therapy session.

Additionally, in some embodiments, the data stored in the database may also serve as a source of data inputs for the therapy note evaluation engine 130. For example, data stored in the database 120 may be queried by the therapy note application 110 and processed to build the inputs for the therapy note evaluation engine 130. For example, the data may be queried utilizing a specific query to collect the data of interest for a specific session note to be analyzed. The query may pull all the relevant data for the specific session note and the query may occur at the run time of the therapy note application 110.

In the embodiment of FIG. 1, the system also generally comprises a therapy notes evaluation engine 130. The therapy notes application 110 may communicate with the therapy notes evaluation engine 130, which can either be part of the therapy notes application 110 or a cloud-based service for evaluating the therapy notes. The therapy note evaluation engine may include two sub modules. For example, the therapy note evaluation engine may include a rule-based evaluation engine. Many elements of the therapy note may be provided in a structured format. One of the functions of the therapy note evaluation engine 130 may be to ensure (for example, checking) that the values for the elements of the therapy note are complete and valid. These checks can be programmatically enforced. The rule-based evaluation engine may enforce these checks or ‘rules’ that have been predetermined by a supervisory therapist as key to effective therapy note evaluation. For example, FIG. 2 illustrates an embodiment of a rule-based engine operational method 200. The various rules that are enforced may include the following.

“Checks” Enforcement for Rule-Based Evaluation Engine
1. Client name is present and valid.
2. Therapy date is present and valid.
3. Current Procedural Terminology (CPT) Code is present and valid.
4. Therapy duration is present and valid.
5. Therapy location is present and valid.
6. Therapy participants are identified and appropriately selected.
   a. Check that client is present and named.
   b. Check that therapist is present and named.
   c. Check that supervisory therapist is present and named.
   d. Check that all other participants (e.g., parents or siblings) are present and named.
7. Client symptoms are selected.
8. Client interventions are selected.
9. Client's current clinical status is selected and/or noted.
10. Client's clinical appearance/emotional state is selected and/or noted.
11. Client's response to therapy treatment is selected and/or noted.
12. Inappropriate/inadequate keywords are not present in the therapy note.

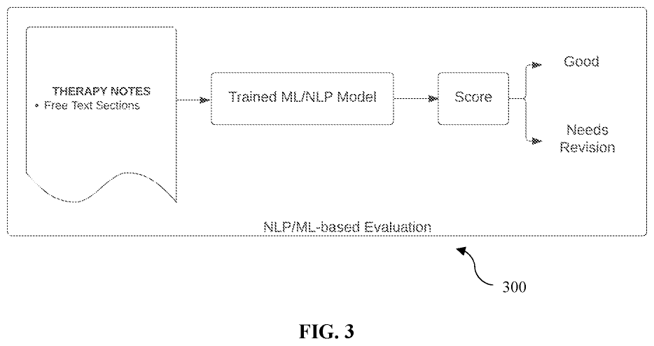

In some embodiments, the therapy note evaluation engine may also include a natural language processing (NLP)/machine earning (ML)-based evaluation engine. FIG. 3 illustrates an embodiment of an NLP/ML-based evaluation engine operational method 300. In addition to structured data fields, the therapy notes also comprise free text fields. The NLP/ML-based evaluation engine may also include an NLP/ML model (such as a therapy notes (TN) model), which may be trained to evaluate each of these free text fields. In some embodiments, the NLP/ML-based evaluation engine may provide a score for each free text field. Based on predefined thresholding associated with the score, the NLP/ML-based engine may be effective to distinguish between acceptable/adequate and unacceptable/inadequate text. In addition to the score, the NLP/ML-based evaluation engine may also provide information on which aspects of the text caused it to deem the input text adequate or not.

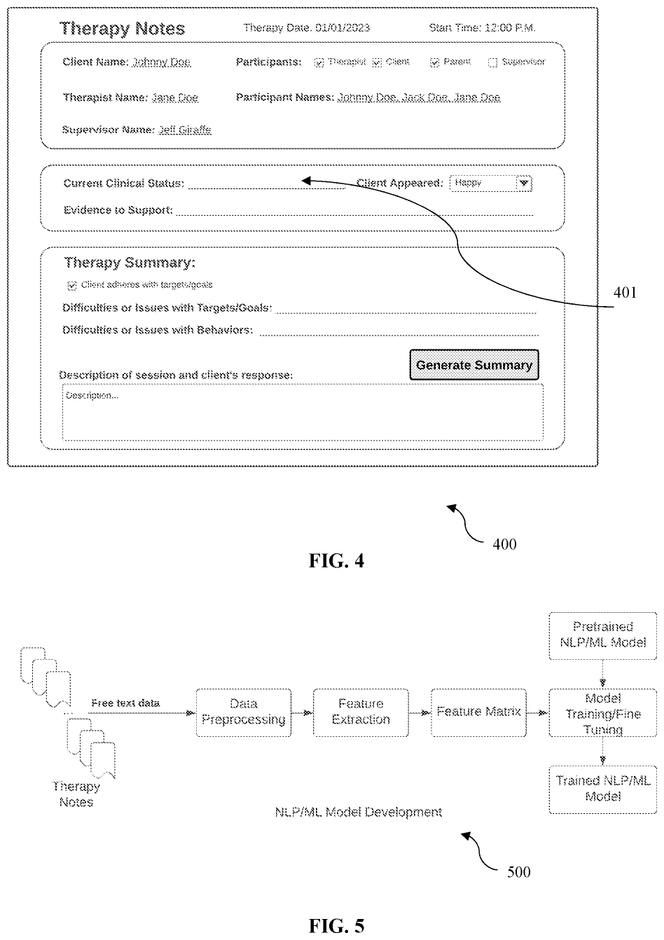

A process for building the NLP/ML-based evaluation engine is further disclosed herein with respect to model development. In some embodiments, multiple NLP/ML models may be trained and deployed for multiple free text fields to be analyzed. An example of an interface 400 for the therapy notes application 110 is illustrated in FIG. 4. For example, a first model may evaluate a therapy narrative and a second model may evaluate an ‘evidence to support’ field under ‘Current Clinical Status’ section 401, as shown in FIG. 4.

In various embodiments, a therapy note may include various elements, for example, various data fields. For instance, data fields present within a therapy note may include, but are not necessarily limited to:
Patient's full name
Date, time (start/end time), length of session
Location of rendered services
Names of session participants
Rendering provider's name
Name of authorized ABA supervisor
Patient's current clinical status
Narrative summaries of session content
ABA techniques attempted, patient's response to treatment, and progress toward treatment goals

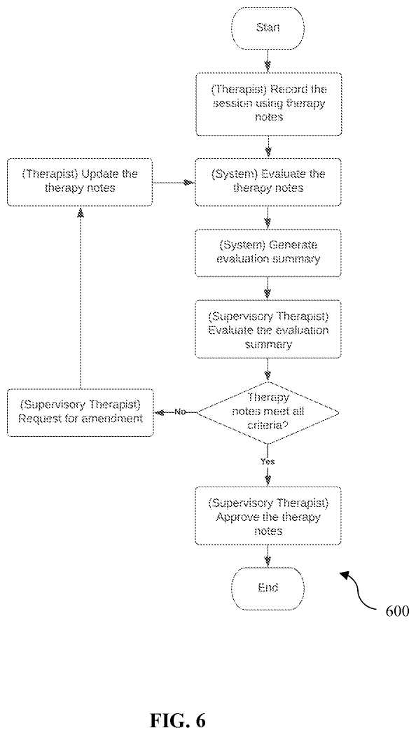

In various embodiments, the system (for example, the system of FIG. 1 or a similar system) may be configured to support various user interactions. FIG. 6 illustrates an embodiment of therapy notes evaluation process 600. As illustrated in FIG. 6, for example, a therapist can interact with the system in order to record details of the treatment session in the therapy notes and/or make changes to the therapy notes, for example, on update requests from a supervisory therapist. Additionally or alternatively, a supervisory therapist can interact with the system in order to evaluate the therapy notes (or, inspect the evaluation of therapy notes made by the system), request changes to the notes from the therapist, and/or approve the notes to be in a completed state. This is detailed in FIG. 6, which illustrates an embodiment of therapy notes evaluation process 600.

In some embodiments, the NLP/ML model may be developed via a suitable process. An example of an NLP/ML model development process 500 is illustrated in FIG. 5. For example, the NLP/ML model development process may start by collecting and processing free text fields from historical therapy notes. The data obtained after processing the therapy notes may be used to extract features. Features selected from the feature extraction step are used to build the final feature matrix that may be used to train the NLP/ML model(s). In some embodiments, publicly-available pretrained models may be selected based on the requirements and retrained/fine-tuned to achieve a desired NLP/ML model having particular characteristics. The detailed description of the steps involved in NLP/ML model development are explained below.

In some embodiments, the NLP/ML model development includes data preprocessing. A variety of data cleaning and preprocessing strategies can be implemented to prepare quality data to train the NLP/ML model and/or for inference. Some of the strategies may include, but are not limited to:
1. Removing punctuations and symbols from the text;
2. Lowering the case of the text;
3. Tokenizing the text into smaller units (i.e., combining groups of words or characters which act as one semantic unit);
4. Removing stopwords (e.g., “my,” “me,” “can,” “we,” “you,” “so,” “then,” etc.);
5. Stemming: Standardizing text where the words are “stemmed” or reduced to their root form (e.g.: “fighting” becomes “fight”); and
6. Lemmatization: Making sure that the words retain their meaning after Stemming (e.g.: “goes” after stemming may be converted to “goe”, whereas “goes” should have been converted to “go”).

In some embodiments, about 80% of the total dataset may be used for training (for example, a training dataset) and the remaining about 20% of the total dataset may be separated for testing (for example, a hold-out test dataset). While in some embodiments, about 80% of the data may be used as a training dataset and about 20% of the dataset may be used as a testing or hold-out dataset, in other embodiments, any suitable proportion of the dataset may be utilized as training and testing dataset. For example, in various embodiments, the training dataset may comprise about 30%, about 35%, about 40%, about 45%, about 50%, about 55%, about 60%, about 65%, about 70%, about 75%, about 80%, about 85%, about 90%, about 95%, or more, and likewise, the hold-out dataset may comprise about 5%, about 10%, about 15%, about 20%, about 25%, about 30%, about 35%, about 40%, about 45%, about 50%, about 55%, about 60%, about 65%, or more. Also, in some embodiments, the hold-out dataset may be divided into two (2), three (3), four (4), five (5), six (6), or more subsets, for example, which may be used to test and/or verify the operation of the NLP/ML model in various iterations. Feature extraction and various steps described below until the final NLP/ML model is obtained may be performed with the training dataset. The hold-out test dataset may only be used to evaluate the final NLP/ML model.

Additionally, in some embodiments, NLP/ML model development includes feature extraction. At the end of the data preprocessing step, list of words may be developed, from which features can be extracted using various methods like Bag of words, TD-IDF, One-hot encoding, Word2Vec, Global Vectors for Word Representation (GloVe), Word Embedding, etc. Large language models (LLMs) require for the text data to be tokenized in using a tokenizer model specific to the LLM. In some embodiments, these methods may be effective to convert the data into numeric form.

Additionally, in some embodiments, NLP/ML model development includes feature matrix development. The output of the feature extraction process may be a vectorized representation of one or more of (for example, each of) the text inputs. In some embodiments, the combination of these vector representations of the input texts (for example, all text input texts) is the feature matrix.

Additionally, in some embodiments, NLP/ML model development includes pretraining a model. Many off-the-shelf machine-learning models, including large language models, are available publicly and may be suitably configured to perform the NLP tasks, as disclosed herein. For example, large language models, also termed as foundation models, are already trained on a large corpus of text data. These models are able to recognize and predict patterns in text. Such pretrained models can be fine-tuned for various tasks and/or various domains downstream. In some embodiments, a suitable NLP/ML model may be chosen based on trial and error.

Additionally, in some embodiments, NLP/ML model development includes transfer learning and fine tuning. For example, many open source off-the-shelf models that have already been trained for some other language task such as text classification, language translation, or text generation are commercially available. Such models may be effective to generate vector representations or encodings of the input texts, but these models may be configured for a different task. Thus, it may be necessary or advantageous to retrain the model for the particular task(s) for which the NLP/ML model may be used. This process may entail modifying the model weights. The process of using the weights learned for a different task and updating them to adapt to a different task is known as transfer learning. The part of this process where the model weights are updated to adapt to a downstream task is known as fine tuning. Fine tuning the model may be accomplished by i) keeping the majority of the model weights frozen and only updating the weights of the final few layers, ii) updating all of the weights in the model or iii) keeping all of the weights frozen and applying additional parameters to train for the downstream task. Any of these three approaches to fine-tuning (for example, methods of fine-tuning) can be used. If a large language model is chosen for the task, techniques such as parameter-efficient fine tuning (PEFT) and low-rank adaptation (LoRA) which fall in the third category of model fine tuning can be used to optimize the fine-tuning process. During fine tuning, the model weights or model parameters are initialized with the pretrained weights and then trained again (fine-tuned) with our dataset.

Additionally, in some embodiments, NLP/ML model development includes hyperparameter optimization. The hyperparameters such as learning rate and regularizations may be optimized. This may be done at the same time as transfer learning.

Additionally, in some embodiments, NLP/ML model development includes prompt engineering. Large language models may be configured for a user to provide the input in the form of language (English or otherwise) and, as such, crafting of a specific prompt can affect the output of the NLP/ML model and its performance on a specific task of interest. As the large language model interprets inputs through the relationships learned between words during the training and fine-tuning process, the prompt utilized may elicit specific responses from the NLP/ML model. If a large language model is chosen for a task, a specific instruction prompt template may be crafted to adapt the base input prompt in order to elicit the most relevant and accurate information from the model. As an example, the text “Please summarize the following information:” may be appended to the beginning of every set of input data. As another example, an example of a session note summary with the instructions “This is an example of a session note summary. [INSERT EXAMPLE SUMMARY] Please perform a similar summarization on the following data:” may be appended to the beginning of every set of input data. This prompt may be crafted in order to maximize the performance of the NLP/ML model at the given task and may be consistent for all data inputs to the NLP/ML model. When finalized, the instruction prompt template may be packaged with the NLP/ML model so prompt engineering happens automatically prior to the data being fed to the NLP/ML model.

Additionally, in some embodiments, NLP/ML model development includes training the NLP/Model. The final NLP/ML model may be trained on the training dataset and may be evaluated on the hold-out test dataset. The NLP/ML model may be deployed as a cloud service or a web service accessible via an application programming interface (API).

In various embodiments, the system 100 may be configured such that the NLP/ML model yields various inferences and/or outputs. For example, whenever a therapy note is completed by a therapist, the free text fields of the therapy note may be preprocessed in the same way as the training data was preprocessed. The preprocessed inputs may be subjected to the same feature extraction method as the training data and the final input vector may be converted into a feature matrix that may be input to the trained NLP/ML model. The trained NLP/ML model may then provide a score for the input text. Based on a preset threshold, the score may be used to determine the quality of the input and output (for example, to the user(s) via the therapy notes application 110). Along with the scores, the therapy notes application 110 may be configured to highlight the areas in the text that led to the NLP/ML model's score, which may also be returned to the user, such as in cases when the quality of the text is deemed low. The determination of the quality of the text may depend on the interpretability of the model chosen. In the case of a non-neural network model, interpretation of text may be a function of feature importance. For a neural network, interpretation of the text may be a function of the areas of activation (or areas of low activation) in the hidden layers of the model.

In various embodiments, training data can be provided with various labels. There are various suitable ways in which the data can be labeled for training an NLP/ML model for the task of evaluating a therapy note. In various embodiments, the output of the NLP/ML model, for example, a numeric score that can be interpreted using a threshold or a discrete label (e.g., good/adequate or bad/inadequate), may depend on the way the training data are labeled. In the first case, where the NLP/ML model is configured to output a numeric score, the labels for the input data may also be set in the same way; for example, the texts in the dataset may be given a score. The range of the scores may be arbitrary. For example, the scores can be floating point numbers between 0 and 1, with 1 representing the highest quality of text; or the scores can be integers between 1 and 10, with 10 being the highest quality of text. This would be characterized as a regression task. In the second case, where the NLP/ML model is configured to output a label directly for the input (for example, where there may still be thresholding but it may be internal to the NLP/ML model), the task may be characterized as a classification task. In this case, the text inputs may be labeled with discrete labels. For example, various data (for example, the text) may be labeled as ‘acceptable’ (or adequate, appropriate) or ‘unacceptable’ (or inadequate, inappropriate).

In the embodiment of FIG. 1, the therapy note application 110 may be configured to provide the capability to receive a therapy note recording and/or other input(s). For example, the therapy note application 110 can provide various capabilities for the therapy notes (e.g., treatment/therapy sessions notes, such as ABA treatment/therapy session notes) to be input/recorded, for example, received by the therapy note application 110 as an audio or video file. Examples of such capabilities and/or methods are described below, although other similar methods and any suitable combination of such methods can be employed.

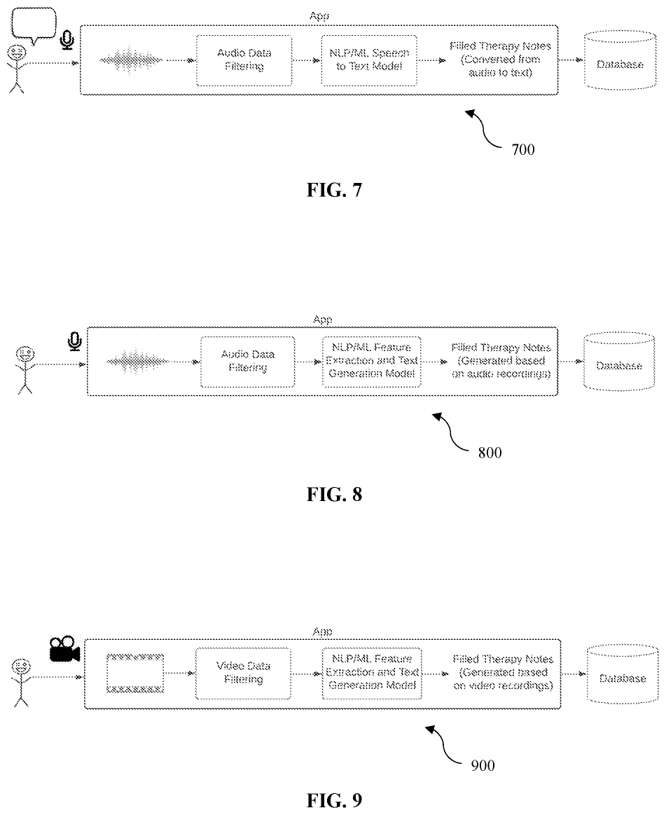

In some embodiments, the therapy notes can be input into the therapy notes application 110 by NLP/ML-based speech to text, audio signal/recording, video signal/recording, manual entry, feature extraction thereof, auto-filling of free text boxes and/or structured fields, and the like, or combinations thereof. For example, the therapy note application 110 may be configured to provide speech to text capabilities. For example, FIG. 7 illustrates an embodiment of a speech to text method 700. This method of input would allow the user (therapist) to dictate the therapy notes, for example the free text sections. The therapy notes application 110 would receive an audio signal (e.g., audio information, audio data) from the caregiver (e.g., therapist) via an audio sensor, for example a microphone. The therapy notes application 110 may be equipped with an NLP/ML-based speech to text capability (for example, via a model such as the NLP/ML model) configured to convert the words spoken by the user (e.g., audio signal, audio information, audio data) into text. The therapy notes application 110 may also be configured to recognize words or groups of words (e.g., sentences or portions thereof, phrases or portions thereof) and pre-select structured fields in the therapy notes, including but not limited to checkboxes, dropdowns and text boxes. The user would review the suggestions presented by the therapy notes application 110 (e.g., text boxes suggestions, free text suggestions, checkbox suggestions, dropdowns suggestions, and the like, or combinations thereof) and decide to either retain, partially revise, or change the suggestions presented by the app, as necessary. Additionally or alternatively, the speech to text feature would also allow the user to select and fill up structured fields in the therapy note including but not limited to checkboxes, dropdowns and text boxes (regardless of whether such structured fields were pre-filled or not with suggestions by the app). The therapy notes application 110 may be configured to implement machine learning to detect various aspects or elements of the session (e.g., various aspects or elements of the speech converted into text) and generate features and text to be suggested in the context of the therapy notes. The machine-learning model may be configured (e.g., by being trained) to convert audio signals (e.g., audio information, audio data) to instructions and/or text. Instructions to select from dropdown menus or check boxes or fill certain sections in the therapy note may be interpreted by the machine-learning model from audio signals. The machine-learning model may also convert the audio signal into text to fill up certain sections in the therapy note. The machine-learning model may also identify keywords from the audio signal (e.g., audio information, audio data) to populate a database containing information about the session. The generated features and text may then be used by the therapy notes application 110 to complete/populate the therapy notes, pending user review.

Additionally or alternatively, the therapy note application 110 may be configured to provide audio and/or video sensing/recording. FIGS. 8 and 9 illustrate an audio input process 800 and a video input process 900, respectively. For example, additionally or alternatively, the therapy notes application 110 may be configured to detect/record audio and/or video from a therapy session and to convert it into features and/or text that may then be used to complete/populate the therapy notes and/or to supplement the therapy notes. For example, the therapy notes application 110 may be configured to detect/record the audio and/or video of the patient and/or other participants of the session (therapist, supervisory therapist, guardian, parent, teacher, etc.) via audio and/or video sensors/devices and to implement machine learning to detect various aspects of the session and generate features and text. In some embodiments, the audio sensor can comprise a microphone, and optionally an amplifier. The generated features and text may be used by the therapy notes application 110 to complete/populate the therapy notes and/or supplement the therapy notes. In some embodiments, the therapy notes application 110 may allow the user to select audio input (alone or in combination with other inputs) and, as a result, the therapy notes application 110 would receive an audio signal (e.g., audio information, audio data, audio recording, etc.). Additionally or alternatively, the therapy notes application 110 would allow the user to select video input (alone or in combination with other inputs) and, as a result, the therapy notes application 110 would receive a video signal (e.g., video information, video data, video recording, etc.). In some embodiments, the therapy notes application 110 may allow the user to select both audio and video as input options for the therapy notes application 110. In other embodiments, the therapy notes application 110 may allow the user to select neither audio nor video as input options for the therapy notes application 110.

In some embodiments, the audio or video sensor(s) can provide for time-series data, e.g., the audio or video sensor(s) gathers time-series data. In other words, the observations (e.g., sensor data) are collected through repeated measurements over time. For example, an input that may be tracked over time comprises physiological data such as heart rate, blood pressure, respiration rate, breathing pattern, oxygen saturation rate, muscle tension level, temperature, one or more electrocardiogram (ECG) features, one or more electromyogram (EMG) features, and the like, or combinations thereof. The sensor data (for example various physiological data) for training can be acquired from a storage device (e.g., smart device and/or cloud storage). At training time, the time-series data can be data collected from a large number of training examples, tracked through the wearable sensor or device. Subsequent to filtering, as disclosed herein, this dataset (e.g., training dataset) can undergo exploratory data analysis to understand the structure/distribution and/or quality of the dataset. Then, the dataset can be corrected or reduced to produce a corrected dataset which excludes outlier data and/or data providing for substantial missingness that may skew the results.

In some embodiments, the audio and/or video signals/recordings can be processed in real-time or near real-time and only transiently stored, as necessary to process the audio and/or video signals/recordings in order to extract features and/or text that would then be used to complete/populate the therapy notes and/or supplement the therapy notes.

Audio and/or video signals/recordings can be filtered as desired to ensure optimal quality for passing into the therapy notes application 110. For example, in some embodiments, prior to data processing and feature engineering to isolate data for input to the therapy notes application 110, the data acquired from the audio and/or video signals/recordings can be manipulated in order to filter noise and/or isolate the vocal signal for each individual in the signal/recording. The signal manipulation and identification, which may be referred to as data “filtering” for purposes of the disclosure herein, can provide for (i) noise (e.g., background noise) removal from the sensor data, wherein the noise may interfere with data extracted for the machine learning-model, and/or (ii) signal isolation associated individual speakers or sound patterns to identify all unique individuals present in the signal/recording.

In an embodiment, one or more digital signal processing (DSP) filters can be constructed by examining the signals (e.g., sensor data) during model development. From the data collected during model development, the magnitude and frequency ranges of the data can be determined, in order to identify the appropriate data frequencies to amplify/pass through the filter (data passband) and the signal frequencies to attenuate (stopband). The stopband frequencies can include the frequency ranges of external noise (e.g., electrical noise, background movement levels, and other relevant high frequency noise) and/or signals associated with other relevant activity (e.g., exercise, sleeping, eating, etc.) that may comprise non-therapy activities.

In an embodiment, the set of passband and stopband frequencies can provide for a series of passive, active, and/or adaptive filters that can be utilized to isolate the therapy session signal. The passive and active filters can include band-pass filters with a stopband that filters (i) high frequency noise due to electrical and/or other interference and (ii) low frequency signals from steady repetitive signals or other non-session signals. The adaptive filters can be utilized to identify and isolate each individual speaker during the recording by changing the filter pattern to focus on one individual's voice or actions. Individual filters can be utilized for each of the distinct features which are being captured by the audio/video recording device. The type of recording device can generally determine the extent of filtering necessary in order to optimize performance and minimize latency of filtering to model prediction. The filtered signal (e.g., filtered sensor data) can be further processed to yield processed sensor data as disclosed herein, wherein the processed sensor data can be input to an ML model (such as the NLP/ML model).

In other embodiments, the audio and/or video signals/recordings can be stored (for example on an internal and/or external memory device), as necessary to process the audio and/or video signals/recordings in order to extract features and/or text that would then be used to complete/populate the therapy notes. In such embodiments, at least a portion of the audio and/or video signals/recordings can be stored in a library and/or database (e.g., on a memory), and at least a portion of the stored information may be used for training an machine-learning model for conversion to therapy notes (e.g., extract features and/or text that would then be used to complete/populate the therapy notes). The memory may include any memory (e.g., memory device) or database module and may take the form of volatile or non-volatile memory. Memory devices may include secondary storage, read-only memory (ROM), random access memory (RAM), input/output (I/O) devices, and network connectivity devices. The audio and/or video signals/recordings can be processed in real-time or near real-time; for example for conversion to therapy notes (e.g., extract features and/or text that would then be used to complete/populate the therapy notes). Additionally or alternatively, the audio and/or video signals/recordings can be processed at a later time (e.g., a convenient time). In yet other embodiments, a first portion of the audio and/or video signals/recordings can be processed in real-time or near real-time and only transiently stored. In such embodiments, a second portion of the audio and/or video signals/recordings can be stored (for example on an internal and/or external memory or memory device), as necessary to process the audio and/or video signals/recordings in order to extract features and/or text that would then be used to complete/populate the therapy notes.

In an aspect, the audio information (e.g., audio signal, audio recording, audio data) can be acquired with a sound sensor (e.g., a sensor configured to detect sound data or information), wherein the sound sensor can transmit sound data to a processor (computing device), and wherein the sound data can comprise vocal sounds produced by the subject, vocal sounds produced by a caregiver, ambient sounds, and the like, or combinations thereof. The vocal sounds may comprise onomatopoeic sounds, words, sentences, sentence portions, phrases, phrase portions, conversations, humming, singing, whispering, yelling, and the like, or combinations thereof. In some embodiments, the sound data can comprise sound pattern data (e.g., pattern of vocal sounds produced by the subject and/or caregiver; sound volume fluctuations; singing vs. speaking vs. humming; etc.).

The therapy notes application 110 may be configured to recognize/identify words or groups of words (e.g., sentences or portions thereof, phrases or portions thereof), sound pattern data, or combinations thereof, and extract features and/or text that would then be used to complete/populate the therapy notes, for example by filling in free text fields and/or pre-select structured fields in the therapy notes, including but not limited to checkboxes, dropdowns and text boxes. The user would review the suggestions presented by the therapy notes application 110 (e.g., text boxes suggestions, free text suggestions, checkbox suggestions, dropdowns suggestions, and the like, or combinations thereof) and decide to either retain, partially revise, or change the suggestions presented by the app, as necessary. Additionally or alternatively, the therapy notes application 110 may be configured to allow the user to select and fill up structured fields in the therapy note including but not limited to checkboxes, dropdowns and text boxes (regardless of whether such structured fields were pre-filled or not with suggestions by the app). The therapy notes application 110 may implement machine learning to detect various aspects or elements of the session (e.g., various aspects or elements of the audio information) and generate features and text to be suggested in the context of the therapy notes.

In an aspect, the video information (e.g., video images, pictures, video recording, video signal, video data) can be acquired with any suitable video recording device, such as a photo camera, a video camera, etc. (e.g., a video recording device configured to detect video image data or information), wherein the video recording device can transmit video image data to a processor (computing device), and wherein the video image data can comprise visually observable elements of the subject, visually observable elements of a caregiver, visually observable elements of the subject's environment, and the like, or combinations thereof.

The visually observable elements may comprise the subject, the caregiver, the subject's spatial position (for example with respect to a caregiver and/or the subject's environment), the caregiver's spatial position (for example with respect to the subject and/or the subject's environment), a body feature thereof (e.g., face, eyes, mouth, hands, limbs, fingers, toes, feet, and the like), movement thereof, or combinations thereof. The visually observable elements may comprise elements of a static picture frame, movements (a series of picture frames) over time, etc. In some embodiments, the video signal/recording data can comprise movement pattern data (e.g., pattern of subject and/or caregiver movements). Movement pattern data may be used to infer behaviors and/or emotions, as well as caregiver response to behaviors and/or emotions.

The therapy notes application 110 may be configured to recognize/identify visually observable elements and extract features and/or text that would then be used to complete/populate the therapy notes, for example by filling in free text fields and/or pre-select structured fields in the therapy notes, including but not limited to checkboxes, dropdowns and text boxes. The user would review the suggestions presented by the therapy notes application 110 (e.g., text boxes suggestions, free text suggestions, checkbox suggestions, dropdowns suggestions, and the like, or combinations thereof) and decide to either retain, partially revise, or change the suggestions presented by the app, as necessary. Additionally or alternatively, the therapy notes application 110 may be configured to allow the user to select and fill up structured fields in the therapy note including but not limited to checkboxes, dropdowns and text boxes (regardless of whether such structured fields were pre-filled or not with suggestions by the app). The therapy notes application 110 may implement machine learning to detect various aspects or elements of the session (e.g., various aspects or elements of the audio information) and generate features and text to be suggested in the context of the therapy notes. The generated features and text may then be used by the therapy notes application 110 to complete/populate the therapy notes, pending user review.

Additionally or alternatively, the therapy notes application 110 may utilize both audio data and video data to extract features and/or text that would then be used to complete/populate the therapy notes, for example by filling in free text fields and/or pre-select structured fields in the therapy notes, including but not limited to checkboxes, dropdowns and text boxes and/or to otherwise supplement the fields present in a therapy note.

In some embodiments, video signals/recordings comprise audio signals/recordings as well, wherein the audio is recorded concurrently with the video.

A variety of machine-learning models, including natural language processing and computer vision models, may be trained to extract a variety of features from the audio and/or video signals (e.g., audio and/or video information, audio and/or video data, audio and/or video recording, etc.). Separate machine-learning models may be configured (for example, by being trained) for specific tasks including but not limited to facial recognition, body movement detection, detection of emotions from the audio and/or video signals. The information extracted from the audio and/or video signals may then be used to generate the contents of the therapy note. Multiple machine-learning models may be configured (e.g., by being trained) to generate specific kinds of features and texts for specific fields and sections in the therapy note. The generated features and text may then be used by the therapy notes application 110 to complete/populate the therapy notes, pending user review.

In some embodiments, multiple machine-learning models may be configured (e.g., by being trained) for object detection; recognizing facial features, body movements, audio volume levels; understanding words being spoken by the participants of the session, for example to identify various things including but not limited to the settings, the participants of the session, the subject/client's mood and emotions throughout the session, the goal that is being worked on, the instruction the subject/client is receiving (from the caregiver, therapist, parent, etc.) to achieve a goal, sentiments (e.g., mood and emotions) of the subject/client towards the various session tasks, overall outcome of the session, etc.; and the like; or combinations thereof. Based on this information extracted by the ensemble of machine-learning models, another machine-learning model can be trained to generate features and/or text required to complete the therapy notes. The generated features may cause the therapy notes application 110 to select from options from dropdown menus and/or checkboxes in the therapy note, as well as fill in structured text fields, as necessary. The generated text may be used to fill free text sections such as the therapy session narrative.

Additionally or alternatively, the therapy note application 110 may be configured to provide for manual entry. Alternatively, the therapy notes application 110 would also allow for recording the therapy note directly in the therapy notes application 110. The user may be able to input the values into the form directly on a mobile device or a personal computer or any other end user device running or otherwise enabling access to the therapy note application 110.

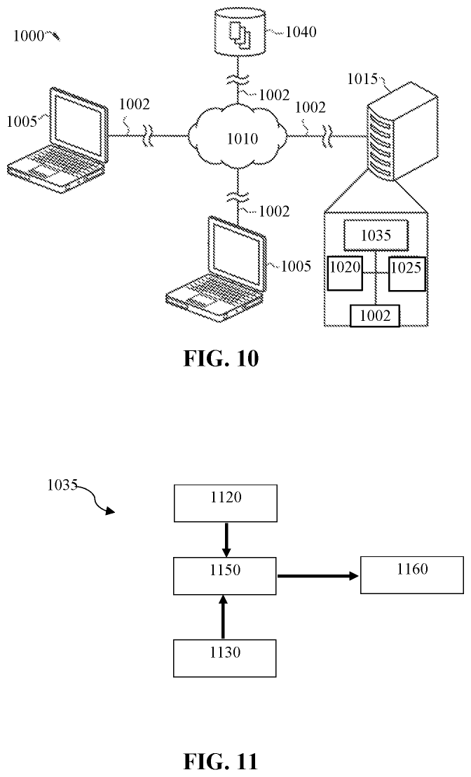

In various embodiments, one or more of the machine-learning models as disclosed herein (for example, the therapy note application 110 or one or more components thereof (e.g., “engines”) may be implemented as a machine-learning model as illustrated in the context of FIG. 10. For example, FIG. 10 illustrates an embodiment of a computing system 1000 that includes a number of clients 1005, a server system 1015, and a data repository 1040 communicably coupled through a network 1010 by one or more communication links 1002 (e.g., wireless, wired, or a combination thereof). The computing system 1000, generally, can execute applications and analyze data received from sensors, such as may be acquired in the performance of the methods disclosed herein. For instance, the computing system 1000 may execute a machine-learning model 1035 to as disclosed herein.

In general, the server system 1015 can be any server that stores one or more hosted applications, such as, for example, the machine-learning model 1035. In some instances, the machine-learning model 1035 may be executed via requests and responses sent to users or clients within and communicably coupled to the illustrated computing system 1000. In some instances, the server system 1015 may store a plurality of various hosted applications, while in other instances, the server system 1015 may be a dedicated server meant to store and execute only a single hosted application, such as the machine-learning model 1035.

In some instances, the server system 1015 may comprise a web server, where the hosted applications represent one or more web-based applications accessed and executed via network 1010 by the clients 1005 of the system to perform the programmed tasks or operations of the hosted application. At a high level, the server system 1015 can comprise an electronic computing device operable to receive, transmit, process, store, or manage data and information associated with the computing system 1000. Specifically, the server system 1015 illustrated in FIG. 10 can be responsible for receiving application requests from one or more client applications associated with the clients 1005 of computing system 1000 and responding to the received requests by processing the requests in the associated hosted application and sending the appropriate response from the hosted application back to the requesting client application.

In addition to requests from the clients 1005, requests associated with the hosted applications may also be sent from internal users, external or third-party customers, other automated applications, as well as any other appropriate entities, individuals, systems, or computers. As used in the present disclosure and as described in more detail herein, the term “computer” is intended to encompass any suitable processing device. For example, although FIG. 10 illustrates a single server system 1015, a computing system 1000 can be implemented using two or more server systems 1015, as well as computers other than servers, including a server pool. The server system 1015 may be any computer or processing device such as, for example, a blade server, general-purpose personal computer (PC), Macintosh, workstation, UNIX-based workstation, or any other suitable device. In other words, the present disclosure contemplates computers other than general-purpose computers, as well as computers without conventional operating systems. Further, the illustrated server system 1015 may be adapted to execute any operating system, including Linux, UNIX, Windows, Mac OS, or any other suitable operating system.

In the illustrated embodiment, and as shown in FIG. 10, the server system 1015 includes a processor 1020, an interface 1030, a memory 1025, and the machine-learning model 1035. The interface 1030 is used by the server system 1015 for communicating with other systems in a client-server or other distributed environment (including within computing system 1000) connected to the network 1010 (e.g., clients 1005, as well as other systems communicably coupled to the network 1010). Generally, the interface 1030 comprises logic encoded in software and/or hardware in a suitable combination and operable to communicate with the network 1010. More specifically, the interface 1030 may comprise software supporting one or more communication protocols associated with communications such that the network 1010 or interface's hardware is operable to communicate physical signals within and outside of the illustrated computing system 1000.

Although illustrated as a single processor 1020 in FIG. 10, two or more processors may be used according to particular needs, desires, or particular embodiments of computing system 1000. Each processor 1020 may be a central processing unit (CPU), a blade, an application-specific integrated circuit (ASIC), a field-programmable gate array (FPGA), or another suitable component. Generally, the processor 1020 executes instructions and manipulates data to perform the operations of server system 1015 and, specifically, the machine-learning model 1035. Specifically, the server's processor 1020 executes the functionality required to receive and respond to requests from the clients 1005 and their respective client applications, as well as the functionality required to perform the other operations of the machine-learning model 1035.

Regardless of the particular implementation, “software” may include computer-readable instructions, firmware, wired or programmed hardware, or any combination thereof on a tangible medium operable when executed to perform at least the processes and operations described herein. Each software component may be fully or partially written or described in any appropriate computer language including C, C++, C #, Java, Visual Basic, assembler, Perl, any suitable version of 4GL, as well as others. It will be understood that while portions of the software implemented in the context of the embodiments disclosed herein may be shown as individual modules that implement the various features and functionality through various objects, methods, or other processes, the software may instead include a number of sub-modules, third-party services, components, libraries, and such, as appropriate. Conversely, the features and functionality of various components can be combined into single components as appropriate. In the illustrated computing system 1000, processor 1020 executes one or more hosted applications on the server system 1015.

At a high level, the machine-learning model 1035 is any application, program, module, process, or other software that may execute, change, delete, generate, or otherwise manage information according to the present disclosure, particularly in response to and in connection with one or more requests received from the illustrated clients 1005 and their associated client applications. In certain cases, only one machine-learning model 1035 may be located at a particular server system 1015. In others, a plurality of related and/or unrelated modeling systems may be stored at a server system 1015, or located across a plurality of other server systems 1015, as well. In certain cases, computing system 1000 may implement a composite hosted application. For example, portions of the composite application may be implemented as Enterprise Java Beans (EJBs) or design-time components may have the ability to generate run-time implementations into different platforms, such as J2EE (Java 2 Platform, Enterprise Edition), ABAP (Advanced Business Application Programming) objects, or Microsoft's .NET, among others. Additionally, the hosted applications may represent web-based applications accessed and executed by clients 1005 or client applications via the network 1010 (e.g., through the Internet).

Further, while illustrated as internal to server system 1015, one or more processes associated with machine-learning model 1035 may be stored, referenced, or executed remotely. For example, a portion of the machine-learning model 1035 may be a web service associated with the application that is remotely called, while another portion of the machine-learning model 1035 may be an interface object or agent bundled for processing at a client 1005 located remotely. Moreover, any or all of the machine-learning model 1035 may be a child or sub-module of another software module or enterprise application (not illustrated) without departing from the scope of this disclosure. Still further, portions of the machine-learning model 1035 may be executed by a user working directly at server system 1015, as well as remotely at clients 1005.

The server system 1015 also includes memory 1025. Memory 1025 may include any memory or database module and may take the form of volatile or non-volatile memory. The illustrated computing system 1000 of FIG. 10 also includes one or more clients 1005. Each client 1005 may be any computing device operable to connect to or communicate with at least the server system 1015 and/or via the network 1010 using a wireline or wireless connection.

The illustrated data repository 1040 may be any database or data store operable to store data, such as data received from a sensor. Generally, the data may comprise inputs to the machine-learning model 1035 and/or output data from the machine-learning model 1035.

The functionality of one or more of the components disclosed with respect to FIG. 10, such as the server system 1015 or the clients 1005, can be carried out on a computer or other device (for example a mobile device) comprising a processor (e.g., a desktop computer, a laptop computer, a tablet, a server, a smartphone, smartwatch, a personal computer, or some combination thereof). Generally, such a computer or other computing device may include a processor (which may be referred to as a central processor unit or CPU) that is in communication with memory devices including secondary storage, read-only memory (ROM), random access memory (RAM), input/output (I/O) devices, and network connectivity devices. The processor may be implemented as one or more CPU chips.

FIG. 11 depicts an example of the operation of the machine-learning model 1035 of FIG. 10. In the embodiment of FIG. 11, the machine-learning model 1035 comprises a machine-learning module 1150 coupled to one or more data stores, for example, data within the data repository 1040. For instance, in the embodiment of FIG. 11, the data within the data repository 1040 of FIG. 10 may include data from a training data store 1120 and/or inputs 1130.

As also shown in FIG. 11, the machine-learning module 1150 can access data, such as data from the training data store 1120, receive inputs, and provide an output 1160 based upon the inputs 1130 and data retrieved from the training data store 1120. Generally, the machine-learning module 1150 utilizes data stored in the training data store 1120, for example, sensor data as disclosed herein, to enable the machine-learning module 1150 to predictively determine the state of a subject based upon additional sensor data evaluated by the machine-learning module 1150.

In some embodiments, at least a portion of the data stored in the training data store 1120 may be characterized as “training data” that is used to train the machine-learning module 1150. As will be appreciated by the ordinarily-skilled artisan upon viewing the instant disclosure, although the Figures illustrate an aspect in which the training data are stored in a single “store” (e.g., at least a portion of the training data store 1120), additionally or alternatively, in some embodiments the training data may be stored in multiple stores in one or more locations. Additionally, in some embodiments, the training data (e.g., at least a portion of the data stored in the training data store 1120) may be subdivided into two or more subgroups, for example, a training data subset, one or more evaluation and/or testing data subsets, or combinations thereof.

Nonlimiting examples of machine-learning models suitable for use in the present disclosure include deep learning model, a generative adversarial network model, a computational neural network model, a recurrent neural network model, a perceptron model, a classical tree-based machine-learning model, a decision tree type model, a regression type model, a classification model, a reinforcement learning model, and combinations thereof. Generally, different machine learning models can be trained and evaluated during the course of building a prediction algorithm, wherein one or more machine learning models can be selected, based on the performance of the model (e.g., best performing models can be selected).

In various embodiments, the systems, methods, and devices disclosed herein may provide several advantages with respect to conventional means of documenting ABA therapy session notes.

The methods disclosed herein present the advantage of increasing workflow efficiency for therapists and/or supervisory therapists, ease of therapy note storage and/or retrieval, automatic and/or programmatic access to therapy notes, error reduction, and the like, or combinations thereof. The therapy notes application 110 can prompt the therapist(s) that certain information that may be necessary and may be desirably included in the therapy notes, for example prior to review of the therapy notes by a supervisory therapist, thereby increasing the efficiency of the supervisory therapist review process, and reducing the numbers of corrections the therapist may be required to make on the therapy notes. In some embodiments, the therapy notes application 110 may be configured to review and/or analyze the sentence structure and suggest changes that would lead to enhanced compliance with the required therapy note format. For example, the free text or narrative section should be prompted by the therapy notes application 110 to include a particular number of sentences including particular subject matter to be associated with certain treatment sessions. As an example, the therapy notes application 110 may suggest that, to be sufficient, a narrative section should include at least two (2), three (3), four (4), or more (complete) sentences for the first two (2) hours of a treatment session and, for each additional hour of treatment, the narrative section should include at least one (1), two (2), or more (complete) sentences. In some embodiments, the therapy notes application 110 may be configured to review/analyze the sentence structure and indicate terms that are “too vague” and/or suggest alternative terms or descriptions that would be more likely to meet the required therapy note format. For example, saying the patient had a “tantrum” is too vague, the therapy notes application 110 may suggest using more descriptive verbs to describe the tantrum, and/or indicate to the user (therapist) that other more descriptive words may be used instead, for example, shouted, screamed, threw the toy, etc. Similarly, for a therapist responding to a tantrum, the therapy notes application 110 may suggest using descriptive verbs to describe the response to the tantrum, and/or indicate to the user (therapist) that other more descriptive words may be used instead, for example, the therapist sang a song to the patient to help them become calmer, the therapist handed a comfort toy to the patient, etc.

In some aspects, an machine-learning model of the type disclosed herein may be embodied in a computing system, the system comprising a means for receiving, by the computing device, a treatment data associated with a subject exhibiting an NDD, the treatment data associated with the subject comprising behavior data, mood data, emotions data, goals data, instruction data, or combinations thereof; and evaluating, by the means, the treatment data associated with the subject via a TN model, wherein the TN model is configured to evaluate the treatment data associated with the subject to generate one or more therapy note features.

Having described various systems and methods herein, certain aspects and advantages of the discloses process(es) system(s), and apparatus can include:

Aspect 1. A method implemented via a computing device, the method comprising receiving, by the computing device, treatment data associated with a subject, the treatment data associated with the subject comprising behavior data, mood data, emotions data, goals data, instruction data, or combinations thereof and evaluating, by the computing device, the treatment data associated with the subject via a therapy notes (TN) model, wherein the TN model is configured to evaluate the treatment data associated with the subject to generate one or more therapy note features.

Aspect 2. The method of Aspect 1, wherein the one or more therapy note features comprise a dropdown menu, a checkbox, a structured text field, a free text section, a therapy session narrative, a content thereof, or combinations thereof.

Aspect 3. The method of one of Aspects 1-2, wherein the treatment data is recorded into a TN application in communication with the computing device.

Aspect 4. The method of Aspect 3, wherein the treatment data is directly recorded into the TN application by a user.

Aspect 5. The method of Aspect 4, wherein the user, responsive to the one or more therapy note features generated by the TN model, modifies the treatment data recorded into the TN application to yield a therapy note.

Aspect 6. The method of Aspect 5, wherein the TN application submits the therapy note for approval, wherein the therapy note is approved or requires revisions.

Aspect 7. The method of Aspect 6, wherein, when the therapy note requires revisions, the TN application sends a notification to the user.

Aspect 8. The method of Aspects 3, wherein the treatment data is received via an audio sensor, a video device, or both.

Aspect 9. The method of Aspect 8, wherein the audio sensor conveys a dictation from a user to the TN application, wherein the dictation comprises at least one word or portion thereof, and wherein the TN model converts the dictation into the one or more therapy note features.

Aspect 10. The method of one of Aspects 8-9, wherein the treatment data acquired via the audio sensor comprises audio data, wherein the audio data comprises vocal sounds produced by the subject, vocal sounds produced by a caregiver, ambient sounds, or combinations thereof, wherein the vocal sounds comprise onomatopoeic sounds, words, sentences, sentence portions, phrases, phrase portions, conversations, humming, singing, whispering, yelling, sound pattern data, pattern of vocal sounds produced by the subject, pattern of vocal sounds produced by a caregiver, sound volume fluctuations, or combinations thereof, and wherein the TN model converts the audio data into the one or more therapy note features.

Aspect 11. The method of one of Aspects 8-10, wherein the treatment data acquired via the video device comprises video data and optionally audio data, wherein the video data comprises visually observable elements of the subject, the subject's spatial position, visually observable elements of a caregiver, the caregiver's spatial position, a body feature thereof, a movement thereof, visually observable elements of the subject's environment, or combinations thereof, and wherein the TN model converts the video data and optionally the audio data, respectively, into the one or more therapy note features.

Aspect 12. The method of one of Aspects 8-11, wherein the one or more therapy note features are recorded into the TN application to yield the therapy notes, wherein a user assesses, in the TN application, the therapy notes, wherein the user revises the therapy notes and/or signals the TN application to submit the therapy notes for approval, and wherein the therapy notes are approved or require revisions.

Aspect 13. The method of Aspect 12, wherein, when the therapy notes require revisions, the TN application sends a notification to the user.

Aspect 14. The method of one of Aspects 1-13, wherein the TN model is a machine-learning model selected from the group consisting of a deep learning model, a generative adversarial network model, a computational neural network model, a recurrent neural network model, a perceptron model, a classical tree-based machine-learning model, a decision tree type model, a regression type model, a classification model, a reinforcement learning model, and combinations thereof.

Aspect 15. The method of one of Aspects 1-14, wherein the subject is characterized as having as a neurodevelopmental disorder (NDD), and wherein the NDD comprises disorders on the autism spectrum or autism spectrum disorder (ASD); attention deficit hyperactivity disorder (ADHD), other specified ADHD, unspecified ADHD; motor disorders, developmental coordination disorder, stereotypic movement disorder, tic disorders, Tourette's disorder or syndrome, persistent (chronic) motor or vocal tic disorder, provisional tic disorder, other specified tic disorder, unspecified tic disorder; cerebral palsy; Rett syndrome; intellectual disabilities, intellectual developmental disorder, global developmental delay, unspecified intellectual disability, unspecified intellectual developmental disorder; communication disorders, language disorder, speech sound disorder or phonological disorder, childhood-onset fluency disorder or stuttering; social or pragmatic communication disorder, unspecified communication disorder; specific learning disorder; other NDDs, other specified NDD, unspecified NDD; or combinations thereof.

Aspect 16. The method of one of Aspects 1-15, wherein the treatment data is derived from treatment provided to the subject, and wherein the treatment comprises ABA therapy, speech therapy, language therapy, physical therapy, occupational therapy, or combinations thereof.

Aspect 17. A system comprising a computing device, the computing device comprising a processor and a non-transitory computer-readable medium, wherein the non-transitory computer-readable medium includes instructions configured to cause the processor to implement a therapy notes (TN) model, wherein the TN model, when implemented via the processor, causes the computing device to receive treatment data associated with a subject having a neurodevelopmental disorder (NDD), the treatment data associated with the subject comprising behavior data, mood data, emotions data, goals data, instruction data, or combinations thereof; and evaluate the data associated with the subject via the TN model, wherein the TN model is configured to evaluate the treatment data associated with the subject to generate one or more therapy note features; and an application in communication with the computing device, wherein the application is configured to receive the one or more therapy note features from the computing device.

Aspect 18. The system of Aspect 17 further comprising a mobile device; wherein the mobile device is selected from the group consisting of a smartphone, a smartwatch, a tablet, a laptop, a personal computer, and combinations thereof; and wherein the application is installed on the mobile device.

Aspect 19. A method implemented via a computing device, the method comprising receiving, by the computing device, training data associated with a plurality of subjects, wherein at least a portion of the subjects are individuals characterized as having a neurodevelopmental disorder (NDD), and wherein the training data associated with each of the plurality of subjects comprise behavior data, mood data, emotions data, goals data, instruction data, or combinations thereof and processing the training data associated with the plurality of subjects to yield a therapy notes (TN) model, wherein the TN model is configured to evaluate treatment data associated with a subject to generate one or more therapy note features.

Aspect 20. The method of Aspect 19, wherein processing the training data associated with the plurality of subjects comprises natural language processing, computer vision, or combinations thereof.

Aspect 21. A method implemented via a computing device, the method comprising generating, by the computing device, one or more therapy note features based upon treatment data associated with subject via a therapy notes (TN) model, the treatment data associated with the subject comprising behavior data, mood data, emotions data, goals data, instruction data, or combinations thereof; and inputting the therapy note features, by the computing device, into a therapy note.

Aspect 22. The method of Aspect 21, wherein the one or more therapy note features comprise a dropdown menu, a checkbox, a structured text field, a free text section, a therapy session narrative, a content thereof, or combinations thereof.

Aspect 23. The method of one of Aspects 21-22, wherein the treatment data is recorded into a TN application in communication with the computing device.

Aspect 24. The method of Aspect 23, wherein the treatment data is directly recorded into the TN application by a user.

Aspect 25. The method of Aspect 24, wherein the user, responsive to the one or more therapy note features generated by the TN model, modifies the treatment data recorded into the TN application to yield the therapy note.

Aspect 26. The method of Aspect 25, wherein the TN application submits the therapy note for approval.

Aspect 27. The method of Aspect 26, wherein, when the therapy note requires revisions, the TN application sends a notification to the user.

Aspect 28. The method of Aspects 23, wherein the treatment data is received via an audio sensor, a video device, or both.

Aspect 29. The method of Aspect 28, wherein the audio sensor conveys a dictation from a user to the TN application, wherein the dictation comprises at least one word or portion thereof, and wherein the TN model converts the dictation into the one or more therapy note features.

Aspect 30. The method of one of Aspects 28-29, wherein the treatment data acquired via the audio sensor comprises audio data, wherein the audio data comprises vocal sounds produced by the subject, vocal sounds produced by a caregiver, ambient sounds, or combinations thereof, wherein the vocal sounds comprise onomatopoeic sounds, words, sentences, sentence portions, phrases, phrase portions, conversations, humming, singing, whispering, yelling, sound pattern data, pattern of vocal sounds produced by the subject, pattern of vocal sounds produced by a caregiver, sound volume fluctuations, or combinations thereof, and wherein the TN model converts the audio data into the one or more therapy note features.

Aspect 31. The method of one of Aspects 28-30, wherein the treatment data acquired via the video device comprises video data and optionally audio data, wherein the video data comprises visually observable elements of the subject, the subject's spatial position, visually observable elements of a caregiver, the caregiver's spatial position, a body feature thereof, a movement thereof, visually observable elements of the subject's environment, or combinations thereof, and wherein the TN model converts the video data and optionally the audio data, respectively, into the one or more therapy note features.

Aspect 32. The method of one of Aspects 28-31, wherein the one or more therapy note features are recorded into the TN application to yield the therapy notes, wherein a user assesses, in the TN application, the therapy notes, wherein the user revises the therapy notes and/or signals the TN application to submit the therapy notes for approval, and wherein the therapy notes are approved or require revisions.

Aspect 33. The method of Aspect 32, wherein, when the therapy notes require revisions, the TN application sends a notification to the user.

Aspect 34. The method of one of Aspects 21-33, wherein the TN model is a machine-learning model selected from the group consisting of a deep learning model, a generative adversarial network model, a computational neural network model, a recurrent neural network model, a perceptron model, a classical tree-based machine-learning model, a decision tree type model, a regression type model, a classification model, a reinforcement learning model, and combinations thereof.

Aspect 35. The method of one of Aspects 21-34, wherein the subject is characterized as having as a neurodevelopmental disorder (NDD), and wherein the NDD comprises disorders on the autism spectrum or autism spectrum disorder (ASD); attention deficit hyperactivity disorder (ADHD), other specified ADHD, unspecified ADHD; motor disorders, developmental coordination disorder, stereotypic movement disorder, tic disorders, Tourette's disorder or syndrome, persistent (chronic) motor or vocal tic disorder, provisional tic disorder, other specified tic disorder, unspecified tic disorder; cerebral palsy; Rett syndrome; intellectual disabilities, intellectual developmental disorder, global developmental delay, unspecified intellectual disability, unspecified intellectual developmental disorder; communication disorders, language disorder, speech sound disorder or phonological disorder, childhood-onset fluency disorder or stuttering; social or pragmatic communication disorder, unspecified communication disorder; specific learning disorder; other NDDs, other specified NDD, unspecified NDD; or combinations thereof.

Aspect 36. The method of one of Aspects 21-35, wherein the treatment data is derived from treatment provided to the subject, and wherein the treatment comprises ABA therapy, speech therapy, language therapy, physical therapy, occupational therapy, or combinations thereof.

Aspect 37. A system comprising a computing device, the computing device comprising a processor and a non-transitory computer-readable medium, wherein the non-transitory computer-readable medium includes instructions configured to cause the processor to implement a therapy notes (TN) model, wherein the TN model, when implemented via the processor, causes the computing device to generate, by the computing device, one or more therapy note features based upon treatment data associated with subject via the TN model, the treatment data associated with the subject comprising behavior data, mood data, emotions data, goals data, instruction data, or combinations thereof; input the therapy note features, by the computing device, into a therapy note; and an application in communication with the computing device, wherein the application is configured to receive the one or more therapy note features from the computing device.

Aspect 38. The system of Aspect 37 further comprising a mobile device; wherein the mobile device is selected from the group consisting of a smartphone, a smartwatch, a tablet, a laptop, a personal computer, and combinations thereof; and wherein the application is installed on the mobile device.

Aspect 39. A method implemented via a computing device, the method comprising receiving, by the computing device, training data associated with a plurality of subjects, wherein at least a portion of the subjects are individuals characterized as having a neurodevelopmental disorder (NDD), and wherein the training data associated with each of the plurality of subjects comprise behavior data, mood data, emotions data, goals data, instruction data, or combinations thereof and processing the training data associated with the plurality of subjects to yield a therapy notes (TN) model, wherein the TN model is configured to generate, by the computing device, one or more therapy note features based upon treatment data associated with subject via the TN model, the treatment data associated with the subject comprising behavior data, mood data, emotions data, goals data, instruction data, or combinations thereof.

Aspect 40. The method of Aspect 39, wherein processing the training data associated with the plurality of subjects comprises natural language processing, computer vision, or combinations thereof.

While embodiments of the disclosure have been shown and described, modifications thereof can be made without departing from the spirit and teachings of the invention. The embodiments and examples described herein are exemplary only, and are not intended to be limiting. Many variations and modifications of the subject matter disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of any patents, patent applications, and publications cited herein are hereby incorporated by reference.

What is claimed is:

1. A method implemented via a computing device, the method comprising:
receiving, by the computing device, treatment data associated with a subject, the treatment data associated with the subject comprising behavior data, mood data, emotions data, goals data, instruction data, or combinations thereof; and
evaluating, by the computing device, the treatment data associated with the subject via a therapy notes (TN) model, wherein the TN model is configured to evaluate the treatment data associated with the subject to generate one or more therapy note features.

2. The method of claim 1, wherein the one or more therapy note features comprise a dropdown menu, a checkbox, a structured text field, a free text section, a therapy session narrative, a content thereof, or combinations thereof.

3. The method of claim 1, wherein the treatment data is recorded into a TN application in communication with the computing device.

4. The method of claim 3, wherein the treatment data is directly recorded into the TN application by a user.

5. The method of claim 4, wherein the user, responsive to the one or more therapy note features generated by the TN model, modifies the treatment data recorded into the TN application to yield a therapy note.

6. The method of claim 5, wherein the TN application submits the therapy note for approval, wherein the therapy note is approved or requires revisions.

7. The method of claim 6, wherein, when the therapy note requires revisions, the TN application sends a notification to the user.

8. The method of claim 3, wherein the treatment data is received via an audio sensor, a video device, or both.

9. The method of claim 8, wherein the audio sensor conveys a dictation from a user to the TN application, wherein the dictation comprises at least one word or portion thereof, and wherein the TN model converts the dictation into the one or more therapy note features.

10. The method of claim 8,
wherein the treatment data acquired via the audio sensor comprises audio data,
wherein the audio data comprises vocal sounds produced by the subject, vocal sounds produced by a caregiver, ambient sounds, or combinations thereof,
wherein the vocal sounds comprise onomatopoeic sounds, words, sentences, sentence portions, phrases, phrase portions, conversations, humming, singing, whispering, yelling, sound pattern data, pattern of vocal sounds produced by the subject, pattern of vocal sounds produced by a caregiver, sound volume fluctuations, or combinations thereof, and
wherein the TN model converts the audio data into the one or more therapy note features.

11. The method of claim 8,
wherein the treatment data acquired via the video device comprises video data and optionally audio data,
wherein the video data comprises visually observable elements of the subject, the subject's spatial position, visually observable elements of a caregiver, the caregiver's spatial position, a body feature thereof, a movement thereof, visually observable elements of the subject's environment, or combinations thereof, and
wherein the TN model converts the video data and optionally the audio data, respectively, into the one or more therapy note features.

12. The method of claim 8,
wherein the one or more therapy note features are recorded into the TN application to yield the therapy notes,
wherein a user assesses, in the TN application, the therapy notes,
wherein the user revises the therapy notes and/or signals the TN application to submit the therapy notes for approval, and
wherein the therapy notes are approved or require revisions.

13. The method of claim 12, wherein, when the therapy notes require revisions, the TN application sends a notification to the user.

14. The method of claim 1, wherein the TN model is a machine-learning model selected from the group consisting of a deep learning model, a generative adversarial network model, a computational neural network model, a recurrent neural network model, a perceptron model, a classical tree-based machine-learning model, a decision tree type model, a regression type model, a classification model, a reinforcement learning model, and combinations thereof.

15. The method of claim 1,
wherein the subject is characterized as having as a neurodevelopmental disorder (NDD), and
wherein the NDD comprises disorders on the autism spectrum or autism spectrum disorder (ASD); attention deficit hyperactivity disorder (ADHD), other specified ADHD, unspecified ADHD; motor disorders, developmental coordination disorder, stereotypic movement disorder, tic disorders, Tourette's disorder or syndrome, persistent or chronic motor or vocal tic disorder, provisional tic disorder, other specified tic disorder, unspecified tic disorder; cerebral palsy; Rett syndrome; intellectual disabilities, intellectual developmental disorder, global developmental delay, unspecified intellectual disability, unspecified intellectual developmental disorder; communication disorders, language disorder, speech sound disorder or phonological disorder, childhood-onset fluency disorder or stuttering; social or pragmatic communication disorder, unspecified communication disorder; specific learning disorder; other NDDs, other specified NDD, unspecified NDD; or combinations thereof.

16. The method of claim 1,
wherein the treatment data is derived from treatment provided to the subject, and
wherein the treatment comprises ABA therapy, speech therapy, language therapy, physical therapy, occupational therapy, or combinations thereof.

17. A system comprising:
a computing device, the computing device comprising a processor and a non-transitory computer-readable medium, wherein the non-transitory computer-readable medium includes instructions configured to cause the processor to implement a therapy notes (TN) model, wherein the TN model, when implemented via the processor, causes the computing device to:
receive treatment data associated with a subject having a neurodevelopmental disorder (NDD), the treatment data associated with the subject comprising behavior data, mood data, emotions data, goals data, instruction data, or combinations thereof; and
evaluate the data associated with the subject via the TN model, wherein the TN model is configured to evaluate the treatment data associated with the subject to generate one or more therapy note features; and
an application in communication with the computing device, wherein the application is configured to receive the one or more therapy note features from the computing device.

18. The system of claim 17 further comprising a mobile device; wherein the mobile device is selected from the group consisting of a smartphone, a smartwatch, a tablet, a laptop, a personal computer, and combinations thereof; and wherein the application is installed on the mobile device.

19. A method implemented via a computing device, the method comprising:
receiving, by the computing device, training data associated with a plurality of subjects, wherein at least a portion of the subjects are individuals characterized as having a neurodevelopmental disorder (NDD), and wherein the training data associated with each of the plurality of subjects comprise behavior data, mood data, emotions data, goals data, instruction data, or combinations thereof; and
processing the training data associated with the plurality of subjects to yield a therapy notes (TN) model, wherein the TN model is configured to evaluate treatment data associated with a subject to generate one or more therapy note features.

20. The method of claim 19, wherein processing the training data associated with the plurality of subjects comprises natural language processing, computer vision, or combinations thereof.
